(19) United States
(12) Patent Application Publication
(10) Pub. No.: US 2005/0112656 A1
(43) Pub. Date: May 26, 2005

(54) Method for isolating and purifying nucleic acid, cartridge for isolating and purifying nucleic acid, and kit isolating and purifying nucleic acid

(75) Inventor: Yoshihide Iwaki, Asaka-shi (JP)

Correspondence Address: BIRCH STEWART KOLASCH & BIRCH, PO BOX 747, FALLS CHURCH, VA, 22040-0747, US

(73) Assignee: Fuji Photo Film Co., Ltd., Minami-Ashigara-shi (JP)

(21) Appl. No.: 10/974,681

(22) Filed: Oct. 28, 2004

(30) Foreign Application Priority Data
Oct. 31, 2003 (JP) P.2003-371783
Oct. 6, 2004 (JP) P.2004-293641

(51) Int. Cl. C12Q 1/68; C07H 21/04

(52) U.S. Cl. 435/6; 536/25.4

(57) ABSTRACT

The invention provides a method for isolating and purifying nucleic acids, which comprises: (1) passing a sample solution containing a nucleic acid through a nucleic acid adsorbing porous membrane to adsorb the nucleic acid to the nucleic acid adsorbing porous membrane; (2) passing a washing solution through the nucleic acid adsorbing porous membrane to wash the nucleic acid adsorbing porous membrane while adsorbing the nucleic acid; and (3) passing an elution solution through the nucleic acid adsorbing porous membrane to desorb the nucleic acid from the nucleic acid adsorbing porous membrane, wherein the nucleic acid adsorbing porous membrane is a porous membrane capable of adsorbing the nucleic acid by interaction involving substantially no ionic bond, and a step of drying the nucleic acid adsorbing porous membrane adsorbing the nucleic acid is not included between the washing step (2) and the recovering step (3).

### BACKGROUND OF THE INVENTION

1. Field of the Invention

The present invention relates a method for isolating and purifying nucleic acids, and a cartridge for isolating and purifying the nucleic acids, and a kit for isolating and purifying the nucleic acids, and more particularly, the invention relates to a method for isolating and purifying the nucleic acids from test specimens including the nucleic acids by use of the cartridge and a power pipette, said cartridge receiving the nucleic acid adsorbing porous membrane in a container having at least two openings; a cartridge for isolating and purifying the nucleic acids; and a kit for isolating and purifying the nucleic acids, having the cartridge.

2. Background Art

Various forms of nucleic acids are used in a variety of fields. For example, in the field of recombinant nucleic acid technology, nucleic acids are used in the form of probes, genomic nucleic acids and plasmid nucleic acids.

In the field of diagnostics, nucleic acids are used in various methods. For example, nucleic acid probes are used routinely in the detection and diagnosis of human pathogen. Likewise, nucleic acids are used in the detection of genetic disorders. Nucleic acids are also used in the detection of food contaminants. Further, nucleic acids are used routinely in locating, identifying and isolating nucleic acids of interest for a variety of reasons ranging from genetic mapping to cloning and recombinant expression.

In many cases, nucleic acids are available in extremely small amounts, and thus isolation and purification procedures are laborious and time consuming. These often time consuming and laborious operations are likely to lead to the loss of nucleic acids. In purifying nucleic acids from samples obtained from serum, urine and bacterial cultures, there is a risk of contamination and false positive results.

One of widely known purification method is a method of adsorbing nucleic acids onto surfaces of silicon dioxide, silica polymers, magnesium silicate and the like, followed by the procedures such as washing and desorbing, to carry out purification (for example, Patent Literature 1: Japanese Patent Publication No. 51065/1995). This method is excellent in isolation ability, but not sufficient in simplicity, rapidness, automation, and aptitude to miniaturization, and industrial mass production of adsorbents with identical performance is difficult. Further, there are other drawbacks, such as inconvenience in handling and difficulty in processing into various shapes.

As one of methods for conveniently and efficiently isolating and purifying the nucleic acid, it is reported to use a solution of adsorbing the nucleic acid to a solid phase and use a solution of desorbing the nucleic acid from the solid phase, respectively, and adsorb the nucleic acid of organic high polymer to the solid phase and desorb the nucleic acid of the same therefrom, thereby isolating and purifying nucleic acid (for example Patent Literature 2: Japanease Patent Laid Open No. 2003-128691).

### SUMMARY OF THE INVENTION

An object of the invention is to provide a method for isolating and purifying the nucleic acids, which is excellent in isolation ability, washing efficiency, simplicity, rapidness, automation, and aptitude to miniaturization, using porous membrane enabling mass production with substantially the same isolation ability, and a cartridge for isolation and purification of the nucleic acid, and a kit for isolation and purification of the nucleic acid.

The present inventors have made intensive studies to solve the above mentioned problems. As a result, they have found that, in the method for isolating and purifying the nucleic acid, it is useful to include steps of adsorbing the nucleic acid to and desorbing the same from the porous membrane. Further, they have found that, in the method for isolating and purifying the nucleic acid, by using the porous membrane capable of adsorbing the nucleic acid by interaction involving substantially no ionic bond, using the washing solution of low surface tension to washing of the nucleic acid adsorbing to the porous membrane, and subsequently recovering the washing solution, it is possible to isolate and purify the nucleic acid from test specimens containing the nucleic acid at high yield, high purification, conveniently and rapidly. The invention has been accomplished based on these findings. That is, the invention comprises the followings.

1. A method for isolating and purifying nucleic acid, comprising the steps of:
(1) passing a sample solution containing a nucleic acid through a nucleic acid adsorbing porous membrane to adsorb the nucleic acid to the nucleic acid adsorbing porous membrane;
(2) passing a washing solution through the nucleic acid adsorbing porous membrane to wash the nucleic acid adsorbing porous membrane while adsorbing the nucleic acid; and
(3) passing an elution solution through the nucleic acid adsorbing porous membrane to desorb the nucleic acid from the nucleic acid adsorbing porous membrane,
wherein the nucleic acid adsorbing porous membrane is a porous membrane capable of adsorbing the nucleic acid by interaction involving substantially no ionic bond, and
a step of drying the nucleic acid adsorbing porous membrane adsorbing the nucleic acid is not included between the washing step (2) and the recovering step (3).

2. The method for isolating and purifying nucleic acid as described in the item 1, wherein the washing solution has surface tension of 3.5 mN/m or less.

3. The method for isolating and purifying nucleic acid as described in the item 1 or 2, wherein in the above mentioned each step of (1), (2) and (3), the sample solution containing the nucleic acid, the washing solution or the elution solution is passed through the nucleic acid adsorbing porous membrane under a pressurizing condition.

4. The method for isolating and purifying nucleic acid as described in the item 1 or 2, which comprises:
injecting the sample solution containing the nucleic acid, the washing solution or the elution solution in the above mentioned each step of (1), (2) and (3) into one opening of the cartridge for isolation and purification of nucleic acid, in which said cartridge comprises the nucleic acid adsorbing porous membrane in a container having at least two openings;
passing the injected solutions through the interior of the cartridge pressurized by use of a differential pressure generator connected to one opening of the container; and
discharging the injected solutions out of the other opening of the container.

5. The method for isolating and purifying nucleic acid as described in the item 1 or 2, wherein a number of times of passing the washing solution through the nucleic acid adsorbing porous membrane adsorbing the nucleic acid to wash the nucleic acid adsorbing porous membrane is once.

6. The method for isolating and purifying nucleic acid as described in the item 1 or 2, wherein the steps of passing the washing solution through the porous membrane adsorbing the nucleic acid to wash the nucleic acid adsorbing porous membrane is performed at room temperature.

7. The method for isolating and purifying nucleic acid as described in the item 1 or 2, wherein the nucleic acid adsorbing porous membrane is a porous membrane of an organic polymer having a polysaccharide structure.

8. The method for isolating and purifying nucleic acid as described in the item 7, wherein the porous membrane of the organic polymer having the polysaccharide structure is a porous membrane of a mixture of cellulose acetates having different acetyl values.

9. The method for isolating and purifying nucleic acid as described in the item 8, wherein the mixture of cellulose acetates having different acetyl values is a mixture of cellulose triacetate and cellulose diacetate.

10. The method for isolating and purifying nucleic acid as described in the item 9, wherein a mixing ratio (mass ratio) of cellulose triacetate and cellulose diacetate is 99:1 to 1:99.

11. The method for isolating and purifying nucleic acid as described in the item 8, wherein the mixture of cellulose acetates having different acetyl values is a mixture of cellulose triacetate and cellulose monoacetate.

12. The method for isolating and purifying nucleic acid as described in the item 8, wherein the mixture of cellulose acetates having different acetyl values is a mixture of cellulose triacetate, cellulose diacetate and cellulose monoacetate.

13. The method for isolating and purifying nucleic acid as described in the item 8, wherein the mixture of cellulose acetates having different acetyl values is a mixture of cellulose diacetate and cellulose monoacetate.

14. The method for isolating and purifying nucleic acid as described in the item 7, wherein the porous membrane of the organic polymer having the polysaccharide structure is a porous membrane of the organic material containing the saponified cellulose acetate.

15. The method for isolating and purifying nucleic acid as described in the item 14, wherein the saponification degree of the saponified cellulose acetate is 5% or more.

16. The method for isolating and purifying nucleic acid as described in the item 14, wherein the organic material of the saponified cellulose acetate is an organic material of the saponified mixture of cellulose acetates having different acetyl values.

17. The method for isolating and purifying nucleic acid as described in the item 16, wherein the saponification degree of the saponified mixture of cellulose acetate having different acetyle values is 5% or more.

18. The method for isolating and purifying nucleic acid as described in the item 16, wherein the organic material of the saponified mixture of cellulose acetate having different acetyl values is a saponified mixture of cellulose triacetate and cellulose diacetate.

19. The method for isolating and purifying nucleic acid as described in the item 18, wherein the mixing ratio (mass ratio) of cellulose triacetate and cellulose diacetate is 99:1 to 1:99.

20. The method for isolating and purifying nucleic acid as described in the item 16, wherein the organic material of the saponified mixture of cellulose acetate having different acetyl values is a saponified mixture of cellulose triacetate and cellulose monoacetate.

21. The method for isolating and purifying nucleic acid as described in the item 16, wherein the organic material of the saponified mixture of cellulose acetate having different acetyl values is a saponified mixture of cellulose triacetate, cellulose diacetate and cellulose monoacetate.

22. The method for isolating and purifying nucleic acid as described in the item 16, wherein the organic material of the saponified mixture of cellulose acetate having different acetyl values is a saponified mixture of cellulose diacetate and cellulose monoacetate.

23. The method for isolating and purifying nucleic acid as described in the item 14, wherein the porous membrane of the organic material containing the saponified cellulose acetate is a porous membrane where an average hole diameter after saponification reduces compared with that before saponification.

24. The method for isolating and purifying nucleic acid as described in the item 23, wherein a ratio of the average hole diameter after saponification to the average hole diameter before saponification is 0.8 or less.

25. The method for isolating and purifying nucleic acid as described in the item 1 or 2, wherein the nucleic acid adsorbing porous membrane is a regenerated cellulose.

26. The method for isolating and purifying nucleic acid as described in the item 1 or 2, wherein the nucleic acid adsorbing porous membrane, is a porous membrane obtained by treating an organic material having no hydrophilic group to introduce hydrophilic group.

27. The method for isolating and purifying nucleic acid as described in the item 26, wherein the introduction of the hydrophilic group to the organic material having no hydrophilic group comprises combining a graft polymer chain having hydrophilic group with the organic material having no hydrophilic group.

28. The method for isolating and purifying nucleic acid as described in the item 1 or 2, wherein the nucleic acid adsorbing porous membrane is a porous membrane obtained by coating the organic material having no hydrophilic group with a material having hydrophilic group to introduce hydrophilic group.

29. The method for isolating and purifying nucleic acid as described in the item 28, wherein the material having hydrophilic group is an organic polymer having hydrophilic group.

30. The method for isolating and purifying nucleic acid as described in the item 1 or 2, wherein the nucleic acid adsorbing porous membrane is a porous membrane of inorganic material having hydrophilic group.

31. The method for isolating and purifying nucleic acid as described in the item 1 or 2, wherein the nucleic acid adsorbing porous membrane is a porous membrane obtained by treating an inorganic material having no hydrophilic group to introduce hydrophilic group.

32. The method for isolating and purifying nucleic acid as described in the item 31, wherein the introduction of the hydrophilic group to the inorganic material having no hydrophilic group comprises combining a graft polymer chain having the hydrophilic group with inorganic material having no hydrophilic group.

33. The method for isolating and purifying nucleic acid as described in the item 1 or 2, wherein the nucleic acid adsorbing porous membrane is a porous membrane obtained by coating the inorganic material having no hydrophilic group with a material having hydrophilic group to introduce hydrophilic group.

34. The method for isolating and purifying nucleic acid as described in the item 33, wherein the material having hydrophilic group is an organic polymer having hydrophilic group.

35. The method for isolating and purifying nucleic acid as described in the item 26, 28, 31 or 33, wherein the hydrophilic group is a hydroxyl group.

36. The method for isolating and purifying nucleic acid as described in the item 1 or 2, wherein the nucleic acid adsorbing porous membrane is a porous membrane where the front and back sides are asymmetric.

37. A cartridge for isolating and purifying nucleic acid, comprising a nucleic acid adsorbing porous membrane in a container having at least two openings, the cartridge being used for the method for isolating and purifying a nucleic acid as described in any one of the items 1 to 36.

38. A kit for isolating and purifying a nucleic acid, which comprises: a cartridge for isolating and purifying the nucleic acid, comprising a nucleic acid adsorbing porous membrane in a container having at least two openings; and a reagent containing at least a washing solution and an elution solution, the kit being used for the method for isolating and purifying a nucleic acid as described in any one of the items 1 to 36.

According to the method for isolating and purifying the nucleic acid of the invention, using, as the porous membrane, the porous membrane capable of adsorbing the nucleic acid by interaction involving substantially no ionic bond, washing the nucleic acid adsorbed to the porous membrane, and subsequently recovering the nucleic acid not passing a drying process of the porous membrane, it is possible to isolate and purify the nucleic acid from the sample specimen containing the nucleic acid conveniently, rapidly and automatically. Further, if using the washing solution of low surface tension, purifying ability may be heightened.

### BRIEF DESCRIPTION OF THE DRAWING

FIG. 1 is a perspective view showing one embodiment of a cartridge for isolation and purification of nucleic acid.

FIG. 2 is a view obtained by electrophoresing the sample solution of the recovered nucleic acid and the size marker λ HIND III.

### DETAILED DESCRIPTION OF THE INVENTION

The method for isolating and purifying the nucleic acid of the invention includes at least (1) the step of passing the sample solution containing the nucleic acid through the nucleic acid adsorbing porous membrane, and adsorbing the nucleic acid to the interior of the porous membrane, (2) the step of washing the nucleic acid adsorbing porous membrane under the condition of adsorbing the nucleic acid, and (3) the step of passing the elution solution through the nucleic acid adsorbing porous membrane so as to desorb the nucleic acid from the interior of the porous membrane.

Preferably, each of the above steps (1), (2), and (3) passes under pressure the sample solution containing the nucleic acid, the washing solution or the elution solution through the nucleic acid adsorbing porous membrane, and more preferably, each of the above mentioned each step of (1), (2) and (3), injects the sample solution containing the nucleic acid, the washing solution or the elution solution into one opening of the cartridge receiving the nucleic acid adsorbing porous membrane in the container having at least two openings, makes the interior of the cartridge pressurized by use of a differential pressure generator connected to said one opening of the cartridge, and passes each of the injected solutions, and dischargs out of the other opening. By passing under pressure the sample solution containing the nucleic acid, the washing solution or the elution solution through the nucleic acid adsorbing porous membrane, the apparatus can be preferably automatized. Pressure is effected preferably at around 10 to 200 kpa, more preferably 40 to 100 kpa.

By the way, the method for isolating and purifying the nucleic acid of the invention may be carried out in particular conveniently and rapidly by use of the cartridge, the kit of the sample solution and an automatic apparatus for isolating and purifying nucleic acid.

In the above mentioned process for isolating and purifying the nucleic acid, it is possible to finish, within 10 minutes, the step from injecting of the sample solution containing the initial nucleic acid to obtaining of the nucleic acid outside of the cartridge within 2 minutes under a desirable condition. Further, it is possible to obtain the nucleic acid at yield of 50 mass % or more in the above process for isolating and purifying nucleic acid, and at yield of 90 mass % or more under the desirable condition.

In addition, in the above process for isolating and purifying the nucleic acid, it is possible to recover the nucleic acid of molecular weight as wide as from 1 to 200 kbp, desirably from 20 to 140 kbp. That is, in comparison with the conventional spin column method using a glass filter, the nucleic acid of long-chain may be recovered.

In the above mentioned process for isolating and purifying the nucleic acid, in regard to absorbance ratio (260 nm/280 nm) of spectra absorbance of 260 nm and 280 nm demanded by an ultraviolet visible spectrophotometer, it is possible to regularly obtain the nucleic acid of high purity of 1.6 to 2.0 in case of DNA and 1.8 to 2.2 in case of RNA, including less impurities. Further, it is possible to recover the nucleic acid having purity around 1.8 in case of DNA and around 2.0 in case of RNA in the absorbance ratio (260 nm/280 nm) of spectra absorbance by the ultraviolet visible spectrophotometer.

In the above process, as the differential pressure generator, there are an injector, pipetter, evaporator, or pumps enabling to pressurize such as a perista pump, otherwise an instrument enabling to reduce pressure such as an evaporator. Among them, the injector is suited to a manual operation, and the pump is good at an automatic operation. The pipetter has a merit of easy one-handed operation. Preferably, the differential pressure generator is detachably combined with the opening of the cartridge for isolating and purifying the nucleic acid.

The test specimens available in this invention are not limited, but apply, for example, in a diagnosing field, to humors such as whole blood, plasma, serum, urine, feces, semen, or saliva, or plants (or their parts), animals (or their parts), bacteria, virus, or their lysates, and solutions prepared from biological materials such as homogenates.

At first, as to these test specimens, cell membranes and nuclear membranes are dissolved, and treated with an aqueous solution (nucleic acid solublizing reagent) containing a reagent solubilizing the nucleic acid. The cell membrane and the nuclear membrane are dissolved thereby, and the nucleic acid disperses into the aqueous solution, and turns out the sample solution containing the nucleic acid.

For dissolving the cell membrane and nuclear membrane and solubilizing the nucleic acids, for example, in case the sample of an object is whole blood, it is necessary to (1) remove red blood cell, (2) remove various proteins, and (3) lyse white blood cell and nuclear membrane. (1) Removal of red blood cell and (2) removal of various proteins are necessary to prevent clogging non-specific adsorption to the membrane and the porous membrane, and (3) lysis of white blood cell and dissolution of nuclear membrane are necessary to solubilize the nucleic acid as an object for extraction.

The test specimen containing the nucleic acid may contain single nucleic acid or different kinds of nucleic acids. A kind of the nucleic acid to be recovered is not especially limited as to DNA or RNA. Number of the test specimen may be one or plural. Length of the test specimen to be recovered is not especially limited, either, for example, the nucleic acids of arbitrary length as several bp to several Mbp may be used. The method for isolating and purifying nucleic acid of this invention can rapidly take out the comparatively long nucleic acid than that of the conventional and convenient method for isolating and purifying nucleic acid, and may be applied to recovering the nucleic acids of preferably more than 50 kbp, more preferably more than 70 kbp.

In the following description, explanation will be made to processes of dissolving cell membranes and nuclear membranes, solubilizing nucleic acids, and obtaining the sample solution containing the nucleic acids from the test specimen. For dissolving cell membranes and nuclear membranes, and solubilizing the nucleic acids, the invention uses a nucleic acid solubilizing reagent. As the nucleic acid solubilizing reagent, chaotropic salt, surface-active agent, and a solution containing proteolytic enzyme may be enumerated.

As the processes of dissolving cell membranes and nuclear membranes, solubilizing the nucleic acids, and obtaining the sample solution containing the nucleic acids from the test specimen, there may be enumerated (I) a process of injecting the test specimen into a container, (II) a process of adding a nucleic acid solubilizing reagent containing the chaotropic salt and the surface-active agent into the container, and mixing the test specimen and the nucleic acid solubilizing reagent, (III) a process of incubating the above obtained mixed solution, and (IV) a process of including the process adding the nucleic acid solubilizing reagent into the incubated mixed solution.

In the processes of dissolving cell membranes and nuclear membranes, solubilizing the nucleic acids, and obtaining the sample solution containing the nucleic acids from the test specimen, an automating treating aptitude is heightened by homogenizing the test specimen. The homogenizing treatment may be carried out by, for example, a supersonic treatment, use of a sharp edged projecting matter, use of high speed agitating treatment, extrusion from a fine space, or use of glass beads.

In the processes of dissolving cell membranes and nuclear membranes, solubilizing the nucleic acids, and obtaining the sample solution containing the nucleic acids from the test specimen, if using the nucleic acid solubilizing reagent containing proteolytic enzyme, the recovering amount and the recovering efficiency of the nucleic acid are increased, and it is possible to make the test specimen containing the necessary nucleic acid fine and rapid.

Proteolytic enzyme may desirably use at least one of serine protease, cystein protease, and metallic protease. Proteolytic enzyme may desirably use a mixture of proteolytic enzyme of more than several kinds.

Serine protease is not especially limited, for example, protease k may be desirably used.

Cystein protease is not especially limited, for example, papain or cathepsins may be desirably used.

Metallic protease is not especially limited, for example, carboxypeptidase may be desirably used.

Proteolitic enzyme may be used in the concentration of preferably 0.001 to 10 IU, more preferably 0.01 to 1 IU per 1 ml of total capacity of reaction series when adding.

Further, the proteolytic enzyme can preferably use a proteolytic enzyme not containing the nucleic acid solubilizing reagent, and can preferably use the proteolytic enzyme containing a stabilizer. As the stabilizer, metallic ion can be preferably used. Actually, magnesium ion is desirable, and can be added, for example, in a form of magnesium chloride. Stabilization of proteolytic enzyme is desirable, and can be added, for example, in a form of magnesium chloride. If containing the stabilizer of proteolytic enzyme, proteolytic enzyme requested to recovering of the nucleic acid can be made fine, and cost requested to recovering nucleic acid can be reduced. It is desirable to contain the stabilizer of proteolytic enzyme preferably in the concentratioin of 1 to 1000 mM, more preferably 10 to 100 mM to all the amount of reaction group.

It is also sufficient that the proteolytic enzyme is in advance mixed together with other reagents as chaotropic salt or surface-active agent, and is applied to recovering of the nucleic acid as one of reagents.

It is also sufficient that the proteolytic enzyme is mixed as more than two reagents different from other reagents as the chaotropic salt or surface-active agent. In the latter case, the reagent containing proteolytic enzyme is firstly mixed with the reagent, followed by mixing the reagent containing chaotropic salt and/or surface-active agents. Otherwise, the reagent containing chaotropic salt or surface-active agent is firstly mixed, followed by mixing the proteolytic enzyme.

Further, the proteolytic enzyme may be directly dropped as an eye lotion into the mixed solution containing the reagent, or the reagent, the chaotropic salt and/or surface-active agent. In this case, the operation may be simplified.

Nucleic acid solubilizing reagent is also preferably supplied under a dried condition. It is possible to use a container previously containing the proteolytic enzyme dried under a frozen and dried condition. It is also possible to obtain the sample solution by using both of the nucleic acid solubilizing reagent supplied under the dried condition and the container previously containing the proteolytic enzyme under the dried condition.

In case of obtaining the sample solution containing nucleic acid by the above mentioned method, the preserving stability of the nucleic acid solubilizing reagent and the proteolytic enzyme is good, and the operation may be simplified without changing the nucleic acid yielding amount.

There is no especial limit to a method of mixing the test specimen, and chaotropic salt and/or nucleic acid solubilizing reagent containing surface-active agent.

When mixing, the agitator desirably carries out mixing 30 to 3000 rpm for 1 second to 3 minutes. It is thereby possible to increase the yield of isolated and purified nucleic acid. It is also desirable to carry out the mixing by overturnings and blendings 5 to 30 times. The mixing may also depend on pipetting operations 10 to 50 times. This case can increase the yield of isolated and purified nucleic acid by an easy operation.

If the solution of mixing the test specimen, and chaotropic salt and/or the nucleic acid solubilizing reagent solution containing surface-active agent is incubated at an optimum temperature of the proteolytic enzyme and for reacting time thereof, the yield of isolated and purified nucleic acid can be increased. The incubation temperature is ordinarily 20 to 70° C., preferably the optimum temperature of the proteolytic enzyme, and the incubation time is ordinarily 1 to 90 minutes, preferably the optimum time of the proteolytic enzyme. The incubation method is not especially limited but carried out by putting in a hot bath or a heater.

In the process of dissolving the cell membrane and nucleic membrane, and solubilizing the nucleic acid so as to provide the sample solution containing nucleic acid from the test specimen, the nucleic acid solubilizing reagent solution containing surface-active agent and chaotropic salt is preferably pH 5 to 10, more preferably pH 6 to 9, further preferably pH 7 to 8.

In the process of dissolving the cell membrane and nucleic membrane, and solubilizing nucleic acid so as to provide the sample solution containing nucleic acid from the test specimen, the concentration in the nucleic acid solubilizing reagent solution of chaotropic salt is preferably more than 5 mol/L, more preferably 0.5 to 7 mol/L, and further preferably 2 to 6 mol/L. As the chaotropic salt, guanidine chloride is preferable, and other chaotropic salts (guanidine isothiocyanate, guanidine thiocyanate, sodium isothiocyanate, potassium iodide, and sodium iodide) may be employed. Instead of a chaotropic salt, it is also possible to use urea as a chaotropic substance. These may be single or used in combination.

In the process of dissolving the cell membrane and nucleic membrane, and solubilizing the nucleic acid so as to provide the sample solution containing nucleic acid from the test specimen, the surface-active agent to be mixed together with the chaotropic salt and/or proteolytic enzyme in the test specimen is, for example, nonion surface-active agent, cation surface-active agent, anion surface-active agent, or ampholytic surface-active agent.

This invention preferably uses nonion surface-active agent. As nonion surface-active agent, it is possible to use polyoxyethylene alkyl phenyl ether based surface-active agent, polyoxyethylene alkyl ether based surface-active agent, or fatty acid Alkanol amide, and desirably use polyoxyethylene alkyl ether based surface-active agent. More preferably, polyoxyethylene alkyl ether based surface-active agent is a polyoxyethylene alkyl ether based surface-active agent selected from POE decyl ether, POE lauryl ether, POE tridecil ether, POE alkylenedecyl ether, POE sorbitan mono laurate, POE sorbitan mono oleate, POE sorbitan mono stearate, tetraolein acid polyoxyethylene sorbitol, POE alkylamine, and POE acetylene glycol.

Further, cationic surface-active agent may be also preferably used. More preferably, cationic surface-active agent is a cationic surface-active agent selected from cetyl trimethyl ammonium promid, dodecyl trimethyl ammonium chloride, tetradecyl trimethyl ammonium chloride, and cetyl pyridinium chloride. These surface-active agents may be single or used in combination.

The concentration in the nucleic acid solubilizing reagent of these surface-active agents is preferably 1 to 20 mass %.

The above mentioned solution of nucleic acid solubilizing reagent may contain a water soluble organic solvent. As the water soluble organic solvent, alcohol is desirable. Alcohol is sufficient with any of a first-class, second-class or third class alcohol. It is possible to desirably use methyl alcohol, ethyl alcohol, propyl alcohol, and their isomers, butyl alcohol and its isomer. These water soluble organic solvent may be single or used in combination. The concentration in the nucleic acid solubilizing reagent of these water soluble organic solvents is preferably 1 to 20%.

When recovering nucleic acids other than DNA or RNA, in the process of dissolving the cell membrane and nucleic membrane, and solubilizing the nucleic acid so as to provide the sample solution containing the nucleic acid from the test specimen, it is preferable to add RNA decomposing enzyme to the solution of nucleic acid solubilizing reagent. In this case, interference by RNA co-existing in the recovered nucleic acid can be reduced. Addition of DNA decomposing enzyme inhibitor is also desirable.

On the other hand, when recovering nucleic acids other than RNA or DNA, it is preferable to add DNA decomposing enzyme to the solution of nucleic acid solubilizing reagent. As RNA decomposing enzyme inhibitor, an inhibitor specifically inhibiting RNA decomposing enzyme.

RNA decomposing enzyme is not especially limited, for example, RNA specifically decomposing enzyme such as rebonuclease H(RNase H) may be desirably used.

DNA decomposing enzyme is not especially limited, for example, DNA specifically decomposing enzyme such as DNase I may be desirably used.

In the process of dissolving the cell membrane and nucleic membrane, and solubilizing the nucleic acid so as to provide the sample solution containing nucleic acid from the test specimen, it is also preferable to include an anti-foam agent in the sample solution containing nucleic acid. As the anti-foam agent, two components of silicone based anti-foam agent and alcohol based ant-foam agent are desirable, and as the alcohol based anti-foam agent, acetylene glycol based surface-active agent is desirable.

As specific examples of the anti-foaming agents, there are enumerated silicone based anti-foaming agents (for example, silicone oil, dimethyl polysiloxane, silicone emaulsion, modified polysiloxane, or silicone compound), alcohol based anti-foaming agents (for example, acetylene glycol, heptanol, ethyl hexanol, high-class alcohl, or polyoxyalkylene glycol), ether based anti-foaming agents (for example, heptylcellosolve, nonylcellosolve-3-heptyl), fatty based anti-foaming agents (for example, vegetable and animal oils), fatty acid based anti-foaming agents (for example, stearic acid, oleic acid, or palmitic acid), metallic soap based anti-foaming agents (for example, aluminum stearate, or calcium stearate), fatty acid ester based anti-foaming agents (for example, natural wax, or tributyl phosphate), phosphoric acid ester based anti-foaming agents (for example, sodium octyl phosphate) amine based anti-foaming agents (for example, dialumiamine), amide based anti-foaming agents (for example, amide stearate), and other anti-foaming agents (for example, ferric sulfate, or bauxite).

As especially preferable anti-foaming agent, the silicone based anti-foaming agent and the alcohol based anti-foaming agent are combined to use. As the alcohol based anti-foaming agent, the acetyleneglycol based surface-active agent is desirably used.

In the process of dissolving the cell membrane and nucleic membrane, and solubilizing the nucleic acid so as to provide the sample solution containing nucleic acid from the test specimen, for water soluble organic solvent to be added to the incubated mixed solution, alcohol may be desirably used. Alcohol is sufficient with any of a first-class, second-class or third class alcohol. It is possible to desirably use methyl alcohol, ethyl alcohol, propyl alcohol, butyl alcohol and its isomer. These water soluble organic solvent may be single or used in combination. The final concentration in the sample solution containing nucleic acid of water soluble organic solvent is preferably 5 to 90 mass %.

In the process of dissolving the cell membrane and nucleic membrane, and solubilizing nucleic acid so as to provide the sample solution containing nucleic acid from the test specimen, as to the sample solution containing the provided necleic acid, the surface tension is preferably less than 50 mN/m (0.05 J/m²), viscosity is preferably 1 to 1000 mPa-s, and specific gravity is 0.8 to 1.2.

In the following description, explanation will be made to the necleic acid adsorbing porous membrane and adsorbing process employed in this invention. The necleic acid adsorbing porous membrane is that the solution can pass the interior. Herein “the solution can pass the interior” referred to in this invention is meant in that, in case pressure differential is generated between a space to which the membrane contact at its one face and a space to which the membrane contact at another face, the solution can pass the interior of the membrane from the side of the space of high pressure to the side of the space of low pressure, otherwise, in case centrifugal force is applied to the membrane, the solution can pass the interior of the membrane in the direction of the centrifugal force.

The necleic acid adsorbing porous membrane of this invention is characterized by such a porous membrane to which the nucleic acid is adsorbed at interaction of no substantial participation of ion bond. This is meant in that “ionization” is not provided under a using condition at the side of the porous membrane, and it is assumed that the nucleic acid and the porous membrane are attracted each other by changing polarity of circumferences. It is thereby possible to isolate and purify the nucleic acid excellent in isolating ability and at good washing efficiency. Preferably, the nucleic acid adsorbing porous membrane is a porous membrane having hydrophilic group, and it is assumed that the hydrophilic group of the nucleic acid and the hydrophilic group of the porous membrane are attracted each other by changing polarity of circumferences. Herein, the porous membrane having the hydrophilic group is meant by such a porous membrane introduced with the hydrophilic group by treating or coating the porous membrane where a material itself forming the porous membrane has the hydrophilic group, or the material forming the porous membrane.

The material forming the porous membrane may be organic or inorganic. As to the nucleic acid adsorbing porous membrane, for example, there are available the porous membrane being the organic material where the material itself forming the porous membrane has the hydrophilic group; the porous membrane where the porous membrane of the organic material not having the hydrophilic group is treated and the hydrophilic group is introduced; the porous membrane where the porous membrane of the organic material not having the hydrophilic group is coated with the material having the hydrophilic group, and the hydrophilic group is introduced; the porous membrane where the material itself forming the porous membrane is the inorganic material having the hydrophilic group; the porous membrane where the porous membrane of the inorganic material not having the hydrophilic group is treated and the hydrophilic group is introduced; and the porous membrane where the porous membrane of the inorganic material not having the hydrophilic group is coated with the material having the hydrophilic group and the hydrophilic group is introduced; and in view of processing easiness, as the material having the porous membrane, the organic material such as an organic polymer is preferably used.

The hydrophilic group designates a polar group (atomic group) having the interaction with water, and is applied to all groups (atomic groups) concerned with adsorption of nucleic acid. As the hydrophilic group, groups of a middle degree in intensity of the interaction with water are desirable (refer to “Groups of not so much intensive in hydrophilic group” in the paragraph of “hydrophilic group”, ENCYCLOPAEDIA CHIMICA issued by Kyoritsu Shuppan Kabushiki Kaisha), for example, hydroxyl group, carboxyl group, and oxyethylene group may be listed. The hydrophilic group is preferably hydroxyl group.

As the porous membranes having hydroxyl group, such porous membranes may be enumerated which are formed with mixtures of polyhydroxethyl acrylic acid, polyhydroxmethaethyl acrylic acid, polyvinyl alcohol, polyvinyl pyrolidone, polyacrylic acid, polymethacrylic acid, polyoxyethylene, acethylcellulose, or acethylcellulose of different acetyl values, and the porous membrane of organic polymer having polysaccharide structure may be desirably used.

As the organic polymers having the polysaccharide structure, desirably available are cellulose, hemi-cellulose, dextran, agarose, dextrine, amylose, amylopectin, starch, glycogen, mannan, glucomannan, lichenan, isolichenan, laminaran, xylan, fructan, alginic acid, hyaluronic acid, chondroitin, chitin, or chitosan, and as far as having polysaccharide structures or derivative thereof, no limit is made to the above listed materials. Ester derivatives of any of the above polysaccharide structures may be also suitably used. Further, saponified materials of ester derivatives of any of the above polysaccharide structures may be also suitably used.

As the esters of ester derivatives of any of the above polysaccharide structures, it is preferable to select from one or more of carboxylate, nitric ester, sulfate, sulfonate, phosphate, phosphonate, and pyrophosphate. Saponified materials of carboxylate, nitric ester, sulfate, sulfonate, phosphate, phosphonate, and pyrophosphate of any of the above polysaccharide structures may be also suitably used.

As carboxylate, it is preferable to select from one or more of alkylcarbonylester, alkenylcarbonylester, aromatic carbonylester, and aromatic alkylcarbonylester. Saponified materials of alkylcarbonylester, alkenylcarbonylester, aromatic carbonylester, and aromatic alkylcarbonylester may be also suitably used.

As ester group of the above mentioned alkylcarbonylester, it is preferable to select from one or more of acetyl group, propionyl group, butyryl group, valer group, heptanoyl group, octanoyl group, decanoyl group, dodecanoyl group, tridecanoyl group, and hexanoyl group, otherwise ester group having octadecanoyl group. Saponified materials of any of the above polysaccharide structures having the ester group selected from one or more of acetyl group, propionyl group, butyryl group, valer group, heptanoyl group, octanoyl group, decanoyl group, dodecanoyl group, tridecanoyl group, and hexanoyl group, and octadecanoyl group may be also suitably used.

Ester group of the above mentioned alkenyl carbonyl ester is preferably ester group having acrylic group or methacrylic group. Saponified materials of any of the above polysaccharide structures having ester group selected from one or more of acrylic group or methacrylic group may be also suitably used.

The ester group of the above mentioned aromatic carbonyl ester is preferably an ester group having benzoyl group or naphthaloyl group. Saponified materials of any of the above polysaccharide structures having an ester group selected from at least one of the above mentioned benzoyl group and naphthaloyl group may be also suitably used.

As the above mentioned ester, preferably available are nitrocellulose, nitrohemicellulose, nitrodextran, nitro agarose, nitrodextrine, nitroamylose, nitroamylopectin, nitro glycogen, nitropluran, nitromannan, nitroglucomannan, nitro lickenan, nitroisolickenan, nitrolaminaran, nitroxylan, nitrofructan, nitro alginic acid, nitro hyaluronic acid, nitrochondroitin, nitrochitin, or nitrochitosan.

Further, saponified materials of nitrocellulose, nitrohemicellulose, nitrodextran, nitro agarose, nitrodextrine, nitroamylose, nitroamylopectin, nitro glycogen, nitropluran, nitromannan, nitroglucomannan, nitro lickenan, nitroiso lickenan, nitrolaminaran, nitroxylan, nitrofructan, nitro alginic acid, nitro hyaluronic acid, nitrochondroitin, nitrochitin, or nitrochitosan may be suitably used.

As the above mentioned sulfate, preferably available are cellulose sulfuric acid, hemicellulose sulfuric acid, dextran sulfuric acid, agarose sulfuric acid, dextrine sulfuric acid, amylase sulfuric acid, amylopectin sulfuric acid, glycogen sulfuric acid, mannan sulfuric acid, glucomannan sulfuric acid, lichenan sulfuric acid, isolichenan sulfuric acid, laminaran sulfuric acid, xylan sulfuric acid, fructan sulfuric acid, alginic acid sulfate, hyaluronic acid sulfate, chondroitin sulfuric acid, chitin sulfuric acid, or chitosan sulfuric acid. Saponified materials of cellulose sulfuric acid, hemicellulose sulfuric acid, dextran sulfuric acid, agarose sulfuric acid, dextrine sulfuric acid, amylase sulfuric acid, amylopectin sulfuric acid, glycogen sulfuric acid, mannan sulfuric acid, glucomannan sulfuric acid, lichenan sulfuric acid, isolichenan sulfuric acid, laminaran sulfuric acid, xylan sulfuric acid, fructan sulfuric acid, alginic acid sulfate, hyaluronic acid sulfate, chondroitin sulfuric acid, chitin sulfuric acid, or chitosan sulfuric acid are also suitably used.

It is preferable to select the above mentioned sulfonate from more than one of any of alkyl sulfonate, alkenyl sulfonate, aromatic sulfonate, and aromatic alkyl sulfonate. Saponified materials of sulfonate from more than one of any of alkyl sulfonate, alkenyl sulfonate, aromatic sulfonate, and aromatic alkyl sulfonate are also suitably used.

As the above mentioned phosphate, preferably available are cellulose phosphate, hemi-cellulose phosphate, dextran phosphate, agarose phosphate, dextrine phosphate, amylose phosphate, amylopectin phosphate, glycogen phosphate, mannan phosphate, glucomannan phosphate, lichenan phosphate, isolichenan phosphate, laminaran phosphate, xylan phosphate, fructan phosphate, alginic acid phosphate, hyaluronic acid phosphate, chondroitin phosphate, chitin phosphate, or chitosan phosphate. Saponified materials of cellulose phosphate, hemi-cellulose phosphate, dextran phosphate, agarose phosphate, dextrine phosphate, amylose phosphate, amylopectin phosphate, glycogen phosphate, mannan phosphate, glucomannan phosphate, lichenan phosphate, isolichenan phosphate, laminaran phosphate, xylan phosphate, fructan phosphate, alginic acid phosphate, hyaluronic acid phosphate, chondroitin phosphate, chitin phosphate, or chitosan phosphate are also suitably used.

As the above mentioned phosphonate, cellulose phosphonate, hemicellulose phosphonate, dextran phosphonate, agarose phosphonate, dextrine phosphonate, amylase phosphonate, amylopectin phosphonate, glycogen phosphonate, mannan phosphonate, glucomannan phosphonate, lichenan phosphonate, isolichenan phosphonate, laminaran phosphonate, xylan phosphonate, fructan phosphonate, alginic phosphonate, hyaluronic phosphonate, chondroitin phosphonate, chitin phosphonate, or chitosan phosphonate. Saponified materials of cellulose phosphonate, hemicellulose phosphonate, dextran phosphonate, agarose phosphonate, dextrine phosphonate, amylase phosphonate, amylopectin phosphonate, glycogen phosphonate, mannan phosphonate, glucomannan phosphonate, lichenan phosphonate, isolichenan phosphonate, laminaran phosphonate, xylan phosphonate, fructan phosphonate, alginic acid phosphonate, hyaluronic acid phosphonate, chondroitin phosphonate, chitin phosphonate, or chitosan phosphonate are also suitably used.

As the above mentioned pyrophosphate, preferably available are cellulose pyrophosphate, hemicellulose pyrophosphate, dextran pyrophosphate, agarose pyrophosphate, dextrine pyrophosphonate, amylase pyrophosphonate, amylopectin pyrophosphonate, glycogen pyrophosphonate, mannan pyrophosphonate, glucomannan pyrophosphonate, lichenan pyrophosphonate, isolichenan pyrophosphonate, laminaran pyrophosphonate, xylan pyrophosphonate, fructan pyrophosphonate, alginic acid pyrophosphonate, hyaluronic acid pyrophosphonate, chondroitin pyrophosphonate, chitin pyrophosphonate, or chitosan pyrophosphonate. Saponified materials of cellulose pyrophosphate, hemicellulose pyrophosphate, dextran pyrophosphate, agarose pyrophosphate, dextrine pyrophosphonate, amylase pyrophosphonate, amylopectin pyrophosphonate, glycogen pyrophosphonate, mannan pyrophosphonate, glucomannan pyrophosphonate, lichenan pyrophosphonate, isolichenan pyrophosphonate, laminaran pyrophosphonate, xylan pyrophosphonate, fructan pyrophosphonate, alginic acid pyrophosphonate, hyaluronic acid pyrophosphonate, chondroitin pyrophosphonate, chitin pyrophosphonate, or chitosan pyrophosphonate are also suitably used.

As the esters of ester derivatives of any of the above polysaccharide structures, it is possible to use methyl cellulose, ethyl cellulose, carboxy methyl cellulose, carboxy ethyl-carbamoyl ethyl cellulose, hydroxy methyl cellulose, hydroxy ethyl cellulose, hydroxylpropyl cellulose, hydroxy propyl methyl cellulose, hydroxy ethyl cellulose, cyano ethyl cellulose, or carbamoyl ethyl cellulose, but no limit is made thereto. Preferably, it is possible to use hydroxy methyl cellulose and hydroxy ethyl cellulose.

Hydroxyl groups of any of the above mentioned polysaccharide structures halogenated at arbitrary degree of substitution are also used.

As the porous membrane of the organic polymer having the polysaccharide structure, acetyl cellulose is desirable, and further it is possible to use the porous membrane of the organic polymer composed of a mixture of acetyl celluloses being different in acetyl value. As the mixture of acetyl celluloses being different in acetyl values, it is possible to use the mixture of triacetyl cellulose and diacetyl cellulose, the mixture of triacetyl cellulose and monoacetyl cellulose, the mixture of triacetyl cellulose, diacetyl cellulose and monoacetyl cellulose, the mixture of diacetyl cellulose and monoacetyl cellulose. In particular, the mixture of triacetyl cellulose and diacetyl cellulose can be preferably used. The mixing ratio (mass ratio) of triacetyl cellulose and diacetyl cellulose is preferably 90:10 to 50:50.

As especially desirable porous membrane of acetyl cellulose, the porous membrane composed of surface-saponified substance of acetyl cellulose described in Patent Laid Open No. 2003-128691 is taken up. The surface-saponified substance is meant by such substances where acetyl cellulose or a mixture of acetyl cellulose being different in acetyl values is saponification-treated, and it is preferable to use the saponified material of the mixture of triacetyl cellulose and diacetyl cellulose, the saponified material of the mixture of triacetyl cellulose and monoacetyl cellulose, the saponified material of the mixture of triacetyl cellulose, diacetyl cellulose and monoacetyl cellulose, and the saponified material of diacetyl cellulose and monoacetyl cellulose. More preferably, the saponified material of triacetyl cellulose and diacetyl cellulose is used. The mixing ratio (mass ratio) of triacetyl cellulose and diacetyl cellulose is preferably 99:1 to 1:99. More preferably, the mixing ratio of the mixture of triacetyl cellulose and diacetyl cellulose is 90:10 to 50:50. In this case, the amount (density) of the hydroxyl group in the surface of a solid phase can be controlled with degrees (saponification degree) of the saponification-treatment. For increasing the isolating efficiency of nucleic acid, the higher amount (density) of the hydroxyl group is the more preferable. It is desirable that the saponification degree (surface-saponification degree) is more than 5% to less than 100%, and more preferably more than 10% to less than 100%.

As to the nucleic acid adsorbing porous membrane composed of the surface-saponificed substance of the above mentioned acetyl cellulose, it is preferable that an average hole diameter of the porous membrane decreases after the saponification treatment than that before the saponification treatment. It is preferable that the ratio of the average hole diameter after the saponification treatment to that before the saponification treatment is less than 0.8, more preferably less than 0.5.

Herein, the saponification treatment is meant by contacting the acetyl cellulose to the saponification-treating solution (for example, solution of sodium hydroxide). Thereby, the portion of acetyl cellulose contacting the saponification-treating solution becomes a regenerated cellulose, and is introduced with hydroxyl group. The thus regenerated cellulose is different from an original cellulose in points of such as crystallized condition. In this invention, it is desirable to use, as the porous membrane, the porous membrane of the regenerated cellulose.

For changing the saponification degree, it is sufficient to carry out the saponification-treatment by changing the density of sodium hydroxide. The saponification degree can be easily measured by NMR, IR or XPS (for example, determined at a rate of reduction of peak of carboxyl group).

The hydrophilic group is introduced into the porous membrane of the organic material not having the hydrophilic group by bonding graft polymer chain having the hydrophilic group to terminal or side-chain of polymer chain.

As method of bonding the graft polymer chain to the porous membrane of the organic material, there are two methods of chemically bonding porous membrane to the graft polymer chain, and of polymerizing a compound having a double bond polymerizing from a starting point of the porous membrane.

In the method of adhering the porous membrane and the graft polymer chain via the chemical bond, the graft can be provided by using the polymer having a functional group reacting with the porous membrane at the terminal or side-chain of polymer chain, and chemically reacting this functional group and a functional group of the porous membrane. As far as enabling to react with functional groups of the porous membrane, no limit is especially made, and as the functional group reacting with the porous membrane, for example, there are taken up silane coupling group such as alkoxy silane, isocyanate group, amino group, hydroxyl group, carboxyl group, sulfone group, phosphate group, epoxy group, allyl group, methacryloyl group, or acryloyl group.

Especially useful compounds as polymers having the reactively functional groups at the terminal or side-chain of polymer chain, include a polymer having a triarcoxylic group at the polymer terminal, a polymer having an amino group at the polymer terminal, a polymer having a carboxyl group at the polymer terminal, a polymer having an isocyanate group at the polymer terminal, or a polymer having an epoxy group at the polymer terminal. As the polymers used at this time, as far as having the hydrophilic group relative with adsorption of hydrophilic group, no limit is especially made, and there are taken up polyhydroxy ethylacrylic acid, polyhydroxymethyl acrylic acid, and salts thereof, polyvinyl alcohol, polyvinylpyrolidone, polyacrylic acid, polymethacrylic acid, and salts thereof, or polyoxyethylene.

A method of forming the graft polymer chain by polymerizing a compound having the double bond polymerizing from the starting point of the porous membrane is generally called as a surface graft polymerization. The surface graft polymerization designates a method of giving active species to the surface of a substrate by means of plasma irradiation, light irradiation or heating, and bonding compounds having a polymerizing double bond disposed so as to contact the porous membrane with the porous membrane via polymerization.

The compounds useful to form the graft polymer chain bonded to the substrate must have two characteristics of having the polymerizing double bond and the hydrophilic group relative to adsorption with the nucleic acid. As these compounds, as far as having the double bond within the molecule, any of the compounds of polymer, oligomer, and monomer are usable. An especially useful compound is the monomer having the hydrophilic group.

Specific examples of the monomer having the especially useful monomer are, for example, preferably the monomer containing hydroxyl group such as 2-hydroxyethylacrylate, 2-hydroxymethylacrylate, or grycerolemonomethacrylatel. The monomer containing carboxyl group such as acrylic acid or methacrylic acid, or alkali metal salt and amine salt are also preferably used.

As another method of introducing the hydrophilic group into the porous membrane of the organic material not having the hydrophilic group, a material having the hydrophilic group is coated. Materials used to coating are not especially as far as having the hydrophilic group relative to adsorption of the nucleic acid, and in view of working easiness, polymer of organic materials is desirable. As the polymer, there are enumerated polyhydroxyethylacrylic acid, polyhydroxymethylacrylic acid, and salts thereof, polyvinylalcohol, polyvinylpyrolidone, polyacrylic acid, polymethacrylic acid, and salts thereof, polyoxyethylene, acetylcellulose, or mixture of acetylcelluloses being different in acetyl values, and polymers having the polysaccharide structure.

Further, it is also possible to perform the coating of acetylcellulose or mixture of acetylcelluloses being different in acetyl values on the porous membrane of the organic material not having the hydrophilic group, and then perform the saponification-treatment on the coated acetylcellulose or mixture of acetylcelluloses being different in acetyl values. In this case, the saponification is desirably more than around 5%, more desirably more than around 10%.

As the porous membrane being the inorganic material having the hydrophilic group, a porous membrane containing silica compound may be listed. As the porous membrane containing silica compound, a glass filter may be listed. Otherwise, a porous silica membrane may be listed as described in the U.S. Pat. No. 3,058,342. The porous silica membrane may be produced by developing, on the substrate, a cation typed amphiphile having bilayer forming ability, removing the solution from a liquid layer existing on the substrate, thereby to prepare a multilayer membrane of the amphiphile, contacting the multilayer membrane to the solution, and subsequently extracting to remove the multilayer membrane of the amphiphile.

As methods of introducing the hydrophilic group into the porous membrane of the inorganic material not having the hydrophilic group, there are two methods of chemically bonding the porous membrane and the graft polymer chain, and of using the monomer having the hydrophilic group having the double bond in the molecule and polymerizing the graft polymer chain from the starting point of the porous membrane.

In case of chemically bonding the porous membrane and the graft polymer chain, the polymer, as the graft polymer chain, having the functional group reacting with the porous membrane at the terminal or the side-chain is used, a functional group reacting with this functional group is introduced into the inorganic material, and the graft polymer is chemically bonded there. Further, in case of using the monomer having the hydrophilic group having the double bond within the molecule and polymerizing the graft polymer chain from the starting point of the porous membrane, the functional group becoming the starting point at polymerizing the compound having the double bond is introduced into the inorganic material. As the graft polymer having the hydrophilic group and as the monomer having the hydrophilic group having the double bond within the molecule, the graft polymer having the hydrophilic group and the monomer having the hydrophilic group having the double bond within the molecule can be desirably used, said graft polymer and the monomer having been referred to in the method of chemically bonding the porous membrane of the organic material and the graft polymer chain.

As another method of introducing the hydrophilic group into the porous membrane of the inorganic material not having the hydrophilic group, there is a method of coating a material having the hydrophilic group. Materials used to coating are not especially limited as far as having the hydrophilic group relative to adsorption of the nucleic acid, and in view of working easiness, polymer of organic materials is desirable. As the polymer, there are enumerated polyhydroxyethylacrylic acid, polyhydroxymethylacrylic acid, and salts thereof, polyvinyl alcohol, polyvinylpyrolidone, polyacrylic acid, polymathacrylic acid, and salts thereof, polyoxyethylene, acetylcellulose, or mixture of acetyl celluloses being different in acetyl values.

Further, it is also possible to perform the coating of acetylcellulose or mixture of acetylcelluloses being different in acetyl values on the porous membrane of the inorganic material not having the hydrophilic group, and then perform the saponification-treatment on the coated acetylcellulose or mixture of acetylcelluloses being different in acetyl values. In this case, the saponification is desirably more than around 5%, more desirably more than around 10%.

As the porous membrane of the inorganic materials not having the hydrophilic group, there are listed metals such as aluminum, glass, cement, ceramics such as porcelains, or new ceramics, silicone, or porous membranes prepared by processing activated carbon.

The thickness of the nucleic acid adsorbing porous membrane is desirably 10 to 500 μm, more desirably 50 to 250 μm. In regard to easy washing of the nucleic acid adsorbing porous membrane, the thinner, the more desirable.

Further, as the nucleic acid adsorbing porous membrane, the porous membrane of the average hole diameter being 0.9 to 5.0 μm can be employed, and the porous membrane of the average hole diameter being 1.5 to 3.5 μm can be employed. Thereby, an enough surface area can be secured for adsorbing the nucleic acid, and clogging scarcely occurs. The average hole diameter of the porous membrane enabling the solution to pass the interior thereof can be determined by a bubble point method (conforming to ASTMF316-86, JIS K3832).

The nucleic acid adsorbing porous membrane may be the porous membrane where the front and back sides are symmetric or where the front and backsides are asymmetric, but the porous membrane where the front and back sides are asymmetric is desirable. Herein, where the front and back sides are asymmetric designates that the physical property or the chemical property changes from one side of the porous membrane to the other side thereof. As an example of the physical property of the membrane, the average hole diameter is enumerated. As an example of the chemical property of the membrane, the saponification is enumerated.

In case of using the porous membrane of the average hole diameter where the front and backsides are asymmetric in this invention, it is desirable that the average hole diameter changes from largeness to smallness in the direction of the liquid passing. Herein, it is desirable to use the porous membrane having a ratio of a maximum hole diameter to a minimum hole diameter being more than 2, more desirably more than 5. Thereby, an enough surface area can be secured for adsorbing the nucleic acid, and clogging scarcely occurs.

In addition, as the nucleic acid adsorbing porous membrane, the porous membrane of porosity being 50 to 95% can be used, more desirably 65 to 80%. As the nucleic acid adsorbing porous membrane enabling the solution to pass the interior thereof, the porous membrane of the bubble point being 0.1 to 10 kgf/cm² can be used, more desirably 0.2 to 4 kgf/cm².

As the nucleic acid adsorbing porous membrane, it is preferable to use the porous membrane of pressure loss being 0.1 to 100 kPa, so that uniform pressure is provided when being at over pressure. More preferably, it is possible to use the porous membrane of pressure loss being 0.5 to 50 kPa. Herein, the pressure loss is meant by a minimum pressure necessary to pass the water per 100 μm of the membrane thickness.

As the nucleic acid adsorbing porous membrane, it is possible to use the porous membrane of an amount of water permeability, when the water passes at pressure of 1 kg/cm² at 25° C., being 1 to 5000 mL per 1 cm² of membrane for 1 minute, and more preferably to use the porous membrane of the amount of water permeability, when the water passes at pressure of 1 kg/cm² at 25° C., being 5 to 1000 mL per 1 cm² of membrane for 1 minute.

As the nucleic acid adsorbing porous membrane, it is possible to preferably use the porous membrane of an adsorbing amount of the nucleic acid being more than 0.1 μg per 1 mg of membrane, more preferably more than 0.9 μg per 1 mg of membrane.

As the nucleic acid adsorbing porous membrane, it is possible to preferably use the porous membrane of cellulose derivative, when the square porous membrane of 5 mm×5 mm is immersed in a trifluoro acetate of 5 mL, being not dissolved within 1 hour but dissolved within 48 hours, and more preferably use the porous membrane of cellulose derivative, when the square porous membrane of 5 mm×5 mm is immersed in trifluoro acetate of 5 mL, being dissolved within 1 hour, but not dissolved within 48 hours when being immersed in dichloromethane of 5 mL.

In case the nucleic acid adsorbing porous membrane is passed through the sample solution containing the nucleic acid, it is desirable to pass the sample solution from one side to the other side of the nucleic acid adsorbing porous membrane, because the liquid is contacted uniformly to the porous membrane. In case the nucleic acid adsorbing porous membrane is passed through the sample solution containing the nucleic acid, it is desirable to pass the sample solution from a larger side of the hole diameter of the nucleic acid adsorbing porous membrane to a smaller side, in view of less clogging.

A flowing rate when passing the nucleic acid adsorbing porous membrane through the sample containing the nucleic acid is preferably 2 to 1500 μL/sec per unit area (cm²) for taking a contacting time of the liquid to the porous membrane. If the liquid contacting time to the porous membrane is too short, an enough isolating and purifying effects are not provided, and if too long, it is not preferable from the viewpoint of operation. Further, the above mentioned flowing rate is preferably 5 to 700 μL/sec per unit area (cm²).

The nucleic acid adsorbing porous membrane where the solution passes at the interior thereof is enough with one sheet, and may be plural sheets. A plurality of sheets of the nucleic acid adsorbing porous membranes may be all the same or respectively different.

It is possible to preferably use a cartridge for isolation and purification of the nucleic acid receiving the nucleic acid adsorbing porous membrane in a container having at least two openings, said nucleic acid adsorbing porous membrane enabling to pass at the interior thereof as mentioned above. Further, it is possible to preferably use the cartridge for isolation and purification of the nucleic acid receiving the plurality of nucleic acid adsorbing porous membranes in the container having at least two openings, said nucleic acid adsorbing porous membrane enabling to pass at the interior thereof as mentioned above. In this case, the plurality of nucleic acid adsorbing porous membranes received in the container having at least two openings may be all the same or respectively different.

The plurality of nucleic acid adsorbing porous membranes may be a combination of the nucleic acid adsorbing porous membranes of the inorganic and organic materials. For example, the combination of the porous membranes of the glass filter and the regenerated cellulose may be taken up. The plurality of nucleic acid adsorbing porous membranes may be the combination of the nucleic acid adsorbing porous membranes of the inorganic and the non-nucleic acid adsorbing porous membranes organic materials. For example, the combination of the porous membranes of the glass filter and nylon or polysulfone may be listed.

Preferably, the cartridge for isolation and purification of the nucleic acid receives no other members than hydrophilic group as mentioned above in the container having at least two openings. As materials for the above mentioned container, such plastics are usable as polypropylene, polystyrene, polycarbonate, or polyvinyl chloride. Biodegradable members are also preferably used. The above mentioned container may be transparent or colored.

As the cartridge for isolation and purification of the nucleic acid, it is possible to use such a cartridge having distinguishing means individually cartridges for isolating and purifying the nucleic acid. As the distinguishing means individually cartridges for isolating and purifying the nucleic acid, barcode or magnetic tapes are listed.

Further, it is possible to use the cartridge for isolating and purifying nucleic acid, having a structure enabling to easily take out the nucleic acid adsorbing porous membrane from the container having at least two openings.

The nucleic acid can be isolated and purified in the following processes by use of the cartridge for isolating and purifying the nucleic acid, receiving the nucleic acid adsorbing porous membrane in the container, said nucleic acid adsorbing porous membrane enabling to pass respective solutions at the interior thereof.

That is, (a) a process of injecting the sample solution containing the nucleic acid into one opening of the cartridge for isolating and purifying the nucleic acid, comprising the nucleic acid adsorbing porous membrane in the container having at least two openings, said nucleic acid adsorbing porous membrane being capable of passing of a solution through the interior thereof; (b) a process of adsorbing the nucleic acid to the interior of the nucleic acid adsorbing porous membrane by pressurizing within the cartridge for isolating and purifying the nucleic acid by a differential pressure generator connected to the other opening of the cartridge for isolating and purifying the nuclei acid, and passing the sample solution containing the injected nucleic acid through the nucleic acid adsorbing porous membrane, and discharging the sample solution out of the other opening of the cartridge for isolating and purifying the nucleic acid; (c) a process of injecting the washing solution into one opening of the cartridge for isolating and purifying the nucleic acid; (d) a process of washing the nucleic acid adsorbing porous membrane, under the condition of the nucleic acid being adsorbed, by pressurizing within the cartridge for isolating and purifying the nuclei acid by the differential pressure generator connected to the other opening of the cartridge for isolating and purifying the nuclei acid, and passing the injected washing solution through the nucleic acid adsorbing porous membrane, and discharging the washing solution out of the other opening; (e) a process of injecting the elution solution into one opening of the cartridge for isolating and purifying the nucleic acid; and (f) a process of desorbing the nucleic acid from the interior of the nucleic acid adsorbing porous membrane and discharging out of the other opening of the cartridge for isolating and purifying the nucleic acid by pressurizing within the cartridge for isolating and purifying the nuclei acid by the differential pressure generator connected to the other opening of the cartridge for isolating and purifying the nuclei acid, passing the injected elution solution through the nucleic acid adsorbing porous membrane, and discharging out of the other opening;

A kit for isolating and purifying nucleic acid can be made by combining a plurality of reagents containing at least the washing solution and the elution solution with the cartridge for isolating and purifying nucleic acid as mentioned above. The reagent may contain such as nucleic acid-solubilizing reagent, buffering agent, salts and water-soluble organic solvent.

In the following description, the washing process will be explained. By washing, the amount of recovering the nucleic acid and the purity are heightened, and the amount of the test specimen containing the necessary nucleic acid can be made extremely small. If automating the washing and the recovering operation, the operation can be carried out conveniently and rapidly. The washing process is desirably finished once for being rapid. In case the purity is more important, the washings are desirably repeated.

If using the nucleic acid adsorbing porous membrane according to this invention, the washing process can be simplified as the following (1) to (3). (1) Number of times that the washing solution passing the nucleic acid adsorbing porous membrane can be made once, (2) the washing process can be performed at room temperature, and (3) the elution solution can be injected into the cartridge immediately after washing. Using all of (1), (2) and (3) is superior in the operating easiness and rapidness, but any one or two can bring about the good effect. In the conventional method, a drying process has been often necessary for rapidly removing organic solvents contained in the washing solution, but since the nucleic acid adsorbing porous membrane of this invention is thin, this process can be omitted.

The amount of the washing solution in the washing process is desirably more than 2 μl/mm². If the amount of the washing solution is much, the washing effect goes up, but for keeping the operationability and restraining the sample solution from flowing away, being less than 200 μl/mm² is desirable.

In the washing process, the rate of flow for passing the washing solution through nucleic acid adsorbing porous membrane is desirably 2 to 1500 μL/sec per unit area (cm²) of the membrane, more desirably 5 to 700 μL/sec. If lowering the rate of passing flow, the washing is made sufficient as much, but since the rapidness of isolating and purifying nucleic acid is important, the above mentioned range is selected.

In the washing process, the temperature of the washing solution is desirably 4 to 70° C. Further, preferably the solution temperature is room temperature.

In the washing process, the washing may be carried out while giving agitation by mechanical vibration or supersonic to the cartridge for isolating and purifying the nucleic acid.

In the washing process, the washing solution in general does not contain enzyme such as nuclease, but can contain enzyme dissolving contaminant as protein, As the cases may be, DNA dissolving enzyme or RNA dissolving enzyme can be contained. If using the washing solution containing DNA dissolving enzyme, only RNA existing in the specimen can be selectively recovered. Reversely, if using the washing solution containing RNA dissolving enzyme, only DNA existing in the specimen can be selectively recovered.

In the washing process, it is preferable that the washing solution contains a water soluble organic solvent and/or a water soluble salt. The washing solution must have a function washing away impurities in the sample solution adsorbed together with the nucleic acid in the nucleic acid adsorbing porous membrane. Therefore, the washing solution must have a composition not isolating the nucleic acid from the nucleic acid adsorbing porous membrane but isolating impurities therefrom. For this object, since the nucleic acid of the water soluble organic solvent is insoluble, it is suited to isolating other components than the nucleic acid while keeping the nucleic acid. If adding a water soluble salt, an adsorbing effect of the nucleic acid goes up, so that a work of selectively removing unnecessary components is heightened.

As the water soluble organic solvent contained in the washing solution, available are methanol, ethanol, isopropanol, n-isopropanol, butanol, or acetone. Among them, ethanol is preferable. The amount of the water soluble organic solvent contained in the washing solution is preferably 20 to 100 wt %, more preferably 40 to 80 wt %.

On the other hand, the water soluble salt contained in the washing solution is preferably salts of halide, and among them, chloride is preferable. The water soluble salt is preferable monovalent or bivalent cation, in particular, alkali metal salt or alkali-earth metal salt are preferable, and among them, sodium salt and potassium salt are preferable, and sodium salt is most preferable.

In case the water soluble salt is contained in the washing solution, and the concentration is preferably more than 10 mmol/L, and an upper limit is not especially provided if the upper limit is within a range not spoiling the solubility of impurities, and preferably less than 1 mol/L, more preferably less than 0.1 mol/L.

Above all, the water soluble salt is sodium chloride, and especially preferably sodium chloride is contained more than 20 mmol/L.

It is desirable that the water solution does not contain a chaotropic substance. It is thereby possible to decrease possibility of containing the chaotropic substance in the recovering process subsequent the washing process. If the chaotropic substance is mixed during the recovering process, it often hinders enzyme reaction such as PCR reaction, and therefore, taking a subsequent enzyme reaction into consideration, it is ideal that the washing solution does not contain the chaotropic substance. Since the chaotropic substance is corrosive and harmful, if the washing process may be performed without the chaotropic substance, this is very advantageous in safety in test operations and also to an experimenter.

Herein the chaotropic substance is, as mentioned above, urea, guanidine salts, sodium isothiocyanate, sodium iodide, or potassium iodide.

When washing in a conventional method of isolating and purifying nucleic acid, since the washing solution has high wettability to the container as the cartridge, it sometimes remain in the container, and it is mixed in the recovering process following the washing process, and this face causes reduction of purity of the nucleic acid or reaction in a subsequent process. Therefore, when carrying out adsorption or desorption of the nucleic acid using the container as the cartridge, it is important to leave no washing solution in the cartridge not to give influences to the following process.

Therefore, for preventing the washing solution in the washing process from mixing into the elution solution of the following process, and restraining the washing solution to a minimum remaining in the cartridge, if decreasing the surface tension, the wettability of the washing solution and the cartridge goes up, so that the amount of the remaining solution can be checked.

The washing solution used in the washing process desirably has the surface tension of less than 3.5 mN/m (0.0035 J/m²) for rapidly washing, more desirably less than 2.8 mN/m, and still more desirably less than 2.8 mN/m. If using the washing solution of a low surface tension, the amount of the washing solution remaining within the porous membrane can be decreased, and the washing can be speeded up.

If the remaining solution is less, the washing efficiency heightens, and the washing is sufficient with onetime, and also in this point, the operation of isolating and purifying the nucleic acid can be made rapid.

Further, since the wettability is good since the surface tension of the washing solution is low, in addition of fast washing rate, a time can be shortened from finishing of washing by the washing solution of the nucleic acid adsorbing porous membrane adsorbing the nucleic acid until injecting of the elution solution into the cartridge. That is, being within 40 seconds is possible, being within 20 seconds is also possible, and even being 5 seconds is operationable without causing practical obstacle.

Since the amount of the remaining solution can be reduced by using the nucleic acid adsorbing porous membrane of this invention and the washing solution of the above mentioned surface tension, and the washing process can be shifted to the recovering process of the adsorbed nucleic acid without drying the porous membrane adsorbing the nucleic acid, the operating convenience and rapidness can be accomplished also in this regard.

The surface tension is adjusted by adjusting the kind and density of the component of the washing solution, for example, the selection of the kind and adjustment of the amount in the above-mentioned water-soluble organic solvent, the adjustment of density of water-soluble chloride, and a small amount addition of the surface-active agent that was mentioned to be able to add to the above-mentioned nucleic acid-solubilizing reagent in order to decrease the surface tension, are exemplified.

In the conventional isolating and purifying nucleic acid, there has been a problem of contamination to the sample by the washing solution splashing to other materials during the washing process. This kind of contamination in the washing process can be restrained by designing the cartridge for isolating and purifying nucleic acid, receiving the nucleic acid adsorbing porous membrane in the container having two openings and the shape of the waste solution container.

In the following description, the process of desorbing the nucleic acid from the nucleic acid adsorbing porous membrane and recovering it is shown.

In the recovering process, the elution solution can be supplied into the cartridge for isolating and purifying the nucleic acid, attaching the nucleic acid adsorbing porous membrane by use of the tube, pipette, or automatic injecting device, otherwise supplying means having the same function as them. The elution solution is fed from one opening (the opening into which the sample solution containing the nucleic acid is injected), and the supplied elution solution is fed from one opening (the opening into which the sample solution containing the nucleic acid is injected), the cartridge for isolating and purifying the nucleic acid is pressurized at the interior thereof by means of the differential pressure generator (for example, a spuit, injector, vacuum pump, or power pipette) connected to the opening different from said one opening, the elution solution is passed through the nucleic acid adsorbing porous membrane, and is discharged from the opening different from said one opening In addition, the elution solution is supplied from one opening and discharged from the same. It is further possible to supply the elution solution from the opening different from said one opening of the cartridge for isolating and purifying the nucleic acid, into which the sample solution containing the nucleic acid, and discharg therefrom. However, it is superior in the washing efficiency and more suitable to supply the elution solution into one opening of the cartridge for isolating and purifying the nucleic acid, pass through the nucleic acid adsorbing porous membrane, and discharg from the opening different from said one opening.

With respect to the volume of the sample solution containing the nucleic acid prepared from the specimen, the nucleic acid can be desorbed by preparing the volume of the elution solution. The amount of the elution solution containing the isolated and purified nucleic acid is determined by the amount of the specimen to be used. The amount of the elution solution generally used is several tens to several hundreds μl, but in case the amount of the specimen is small, or a much amount of the nucleic acid is isolated and purified, the amount of the elution solution can be changed within a range from 1 to several tens μl.

As the elution solution, a buffer aqueous solution such as preferably a purified distilled water, Tris/EDTA buffer may be used. When supplying the recovered nucleic acid to PCR (polymerase chain reaction), the buffer solution used to PCR (for example, the aqueous solution having the final concentration of KCl 50 mmol/l, Tris-Cl 10 mmol/l, MgCl2 1.5 mmol/l) may be used.

pH of the elution solution is desirably pH 2 to 11, more desirably pH 5 to 9. In particular, ionic strength and salt concentration give effects to elusion of the adsorbed nucleic acid. The elution solution desirably has the ionic strength of less than 290 mmol/L and the salt concentration of less than 90 mmol/L. In such manner, the recovering rate of the nucleic acid goes up, and the nucleic acid can be more recovered. The recovered nucleic acid is sufficient with any of DNA, RNA, one chain, two chains, and straight chain.

By making the volume of the elution solution less than the volume of the sample solution containing the initial nucleic acid, the elution solution containing a concentrated nucleic acid can be obtained. Preferably, (volume of the elution solution): (volume of the sample solution)=1:100 to 99:100, more preferably (volume of the elution solution): (volume of the sample solution)=1:10 to 9:10. Thereby, no operation is required for concentration after the process for isolating and purifying the nucleic acid, and the nucleic acid can be easily concentrated. By these methods, it is possible to provide a method of obtaining the solution of the nucleic acid more concentrated than the sample solution.

As another method, if desorbing the nucleic acid under a condition that the volume of the elution solution is more than the sample solution containing the initial nucleic acid, it is possible to obtain the elution solution containing the nucleic acid of a desired concentration, and obtain an elution solution containing the nucleic acid of a concentration suited a subsequent process (such as PCR). Preferably, (volume of the elution solution):(volume of the sample solution)=1:1=5:1 can be provided. Thereby, such a merit is provided which removes a trouble of adjusting the concentration after isolating and purifying the nucleic acid. Further, if using an enough amount of the elution solution, the rate of recovering the nucleic acid from the porous membrane can be increased.

If changing the temperature of the elution solution in response to objects, the nucleic acid can be easily recovered. For example, if keeping the temperature of the elution solution 0 to 10° C. and desorbing the nucleic acid from the porous membrane, the solution of the nucleic acid can be obtained easily and efficiently by restraining action of the nucleic acid dissolving enzyme to avoid dissolution of the nucleic acid without adding any reagent avoiding dissolution by enzyme or a special operation.

In case the temperature of the elution solution is 10 to 35° C., the nucleic acid can be recovered at general room temperature, and the nucleic acid can be desorbed, isolated and purified without taking any complicated process.

As a further method, if holding the elution solution at high temperature, for example, 35 to 70° C., the desorption of the nucleic acid from the porous membrane can be practiced without taking any complicated operation.

The number of times of injecting the elutions olution is not limited, and once or plural times are enough. In general, for rapidly and easily isolating and purifying the nucleic acid, the recovery is once practiced, but for recovering the much amount of recovering nucleic acid, the elution solution can be injected several times.

In the recovering process, it is possible to keep the elution solution of the nucleic acid having the composition available in a subsequent process. The isolated and purified nucleic acid is often amplified by PCR (polymerse chain reaction). In this case, the solution of the isolated and purified nucleic acid can be diluted by the above mentioned buffer solution suited to PCR method. If using the buffer solution as the elution solution suited to PCR method, preferably, the process can be easily and rapidly shifted to the following PCR method.

In the recovering process, a stabilizer can be added in the elution solution of the nucleic acid for preventing dissolution of the recovered nucleic acid. As the stabilizer, an antibacterial agent, anti-fungal agent or a nucleic acid dissolution inhibitor may be added. As an inhibitor of nuclease, EDTA is taken up. As another embodiment, the stabilizer may be in advance added in the recovering container.

No limit is especially made to the recovering container used in the recovering process, such a recovering container made of no adsorption of 260 nm is available. In this case, the concentration of recovered nucleic acid solution can be measured, not moving to another container. As a raw material of no adsorption of 260 nm, for example, a quartz glass is taken up, but not limit is made thereto.

In the method for isolating and purifying a nucleic acid according to the present invention, adsorption and desorption of nucleic acid is able to be carried out using a cartridge for isolating and purifying a nucleic acid where the above-mentioned solid phase is received in a container preferably having at least two openings.

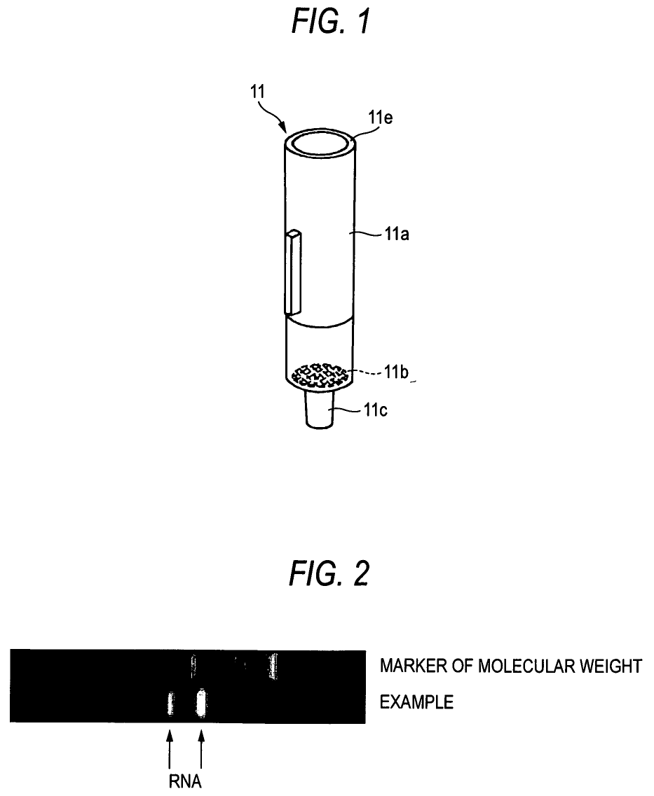

FIG. 1 shows one embodiment of the cartridge for isolating and purifying a nucleic acid of the present invention. In the cartridge 11 for isolating and purifying a nucleic acid of the present invention, the nucleic acid adsorbing porous membrane 11b is held at the bottom of the thecal body 11a having the opening 11e at the upper end thereof, and the lower portion of the thecal body 11a is formed into a funnel shape, and in the lower portion of the thecal body 11a, the discharge portion 11c, which is nozzle-shaped tubule, is formed in a protrusive form. The cartridge 11 for isolating and purifying a nucleic acid in FIG. 1 has a structure, in which the thecal body 11a is divided into the lower portion and the upper portion that can be fitted from each other.

For the isolation and purification of the nucleic acid using the cartridge 11 for isolating and purifying, a nucleic acid, the sample solution, washing solution or elution solution is injected from the opening 11e at the upper end thereof; and the interior of the thecal body 11a is pressurized by use of a differential pressure generator connected to the opening 11e or the discharge portion 11c to pass each solution through the nucleic acid adsorbing porous membrane 11b and to discharge a nucleic acid out of the discharge portion 11c.

There is no particular limitation for a material of the container and anything may be used so far as it is able to receive a solid phase and is able to install at least two openings. In view of easiness in the manufacture, plastic is preferred. It is preferred to use a transparent or semitransparent plastic such as polystyrene, polymethacrylate, polyethylene, polypropylene, polyester, Nylon and polycarbonate.

There is also no particular limitation for the shape of a solid phase received in the above-mentioned container and that may be any shape such as circular, square, rectangular and elliptic and, in the case of membrane, it may be tubular, rolled or beady where an organic macromolecule having hydroxyl group on the surface is coated. In view of a manufacturing aptitude, a shape having high symmetry such as circle, square, cylinder or roll and beads are preferred.

It is preferred that inner volume of a container is determined by the volume of a sample solution to be treated and, usually, it is expressed in terms of volume of a solid phase to be received therein. Thus, it is preferred to be in such a size that one to about six sheet(s) of solid phase where thickness is about 1 mm or less (such as about 50 to 500 μm) and diameter is about 2 mm to 20 mm is/are received.

It is preferred that the end of the side contacting the container of the solid phase is closely adhered to the inner wall surface of the container to such an extent that a sample solution, etc. do not pass.

It is preferred that a side to an opening used as an inlet for a sample solution, etc. (an opening side from the solid phase in the container) from the solid phase of the container having at least two openings is made in such a structure that it not closely adhered to the inner wall of the container but space is placed there so that a sample solution, etc. is diffused to whole surface of the solid phase as uniform as possible.

As the cartridge for isolating and purifying a nucleic acid, a cartridge for isolating and purifying a nucleic acid having a means for identifying each cartridge for isolating and purifying a nucleic acid may be used. As the means for identifying each cartridge for isolating and purifying a nucleic acid, a bar code, magnetic tape or the like is exemplified.

A cartridge for isolating and purifying a nucleic acid having a structure capable of taking out the nucleic acid adsorbing porous membrane from the container having at least two openings may be used.

### EXAMPLES

### Example 1

(1) Production of Nucleic Acid Purifying Cartridge

The container having 7 mm inner diameter for the cartridge of isolating and purifying nucleic acid, having a portion of receiving the nucleic acid adsorbing porous membrane was produced with a high impact polystyrene. As the nucleic acid adsorbing porous membrane of the mixture of two kinds of acetyl celluloses having different acetyl values, the porous membrane having the mixing ratio of triacetyl cellulose and diacetyl cellulose being 6:4 (the membrane thickness=70 μm, and the average hole diameter=1.2 μm) was received in the portion of receiving the nucleic acid adsorbing porous membrane for the above mentioned cartridge. Thus, the cartridge was produced.

(2) Preparation of RNA Solubilized Reagent and Washing Solution

RNA solubilized reagent and the washing solution prescribed in Table 1 were prepared. The surface tension of the washing solution was 3.0 mN/m at 25° C.

TABLE 1

| | |
|---|---|
| (Solution of nucleic acid solubilized reagent) | |
| Guanidine hydrochloride (by Life Technology Inc.) | 382 g |
| Tris (by Life Technology Inc.) | 12.1 g |
| TritonX-100 (by ICN) | 10 g |
| Distilled water | 1000 ml |
| Washing solution | |
| 10 mM Tris-HCl (pH 7.5) | |
| 65% Ethanol | |

(3) Nucleic Acid Isolation-Purification Operation

The isolation-purification operation of the nucleic acid in the cell was performed from the culture solution of the human cancerated bone marrow cell (HL60) in the under mentioned order. That is, the culture solution was sampled such that the cell number would be 1×10⁶, and was subjected to the centrifugal separation for 5 minutes. The cell was precipitated, the supernatant liquid was removed, and the cell was obtained. The above mentioned HL60 cell (1×10⁶) was added with 200 μl of RNA solubilized reagent solution and agitated. Subsequently, 200 μl of ethanol and 20 μl of protease K solution (by SIGMA) were incubated at 60° C. for 10 minutes. After incubation, 200 μl of ethanol was added, and agitated to produce the reagent solution containing RNA.

The reagent solution containing RNA was injected into one opening of the cartridge for nucleic acid adsorbing porous membrane, said cartridge being produced in the above (1) and having the nucleic acid adsorbing porous membrane of the mixture of acetyl celluloses having different acetyl values. Subsequently, the power pipette was connected to said one opening of the cartridge, so that the interior of the cartridge was pressurized, and the sample solution containing the injected RNA was passed through the nucleic acid adsorbing porous membrane, contacted thereto, and discharged out of the other opening of the cartridge.

Next, 500 μL of the washing solution was injected into said one opening of the cartridge for nucleic acid adsorbing porous membrane, and the power pipette was connected to said one opening of the cartridge, so that the interior of the cartridge was pressurized, and the injected washing solution was passed through the nucleic acid adsorbing porous membrane, and discharged out of the other opening of the cartridge.

Then, 200 μL of the elution solution was injected into said one opening of the cartridge for nucleic acid adsorbing porous membrane, and the power pipette was connected to said one opening of the cartridge, so that the interior of the cartridge was pressurized, and the injected elution solution was passed through the nucleic acid adsorbing porous membrane, and discharged out of the other opening of the cartridge.

(4) Confirmation of Isolation-Purification of RNA

The agarose gel electrophoresis was carried out with the elution solution. The result is shown in FIG. 1 where the sample solution of the nucleic acid recovered in Example and the size marker λ HIND III were obtained by the electrophoresis (conditions: by Life Technology Inc., 1 mass % of agarose, 100 V, 30 min.). As is seen from FIG. 1, RNA can be isolated and purified at high recovering efficiency by use of the cartridge and the pressure device, said cartridge having the nucleic acid adsorbing porous membrane of the mixture of acetyl celluloses having different acetyl values.

The purification degree of the nucleic acid obtained in Example 1 is shown with the ratio of spectra absorbance of 260 nm and 280 nm (A260/A280).

TABLE 2

| | A260/A280 |
|---|---|
| Example 1 | 1.826 |

The purification degree shown in Table is very high, and the test specimen of the nucleic acid of high purification could be obtained through the easy operation for about 5 minutes and the easy device.

The same operation was carried out excepting that the washings were done twice continuously for 20 seconds in the washing process in Example 1, and the results of isolating and purifying the nucleic acid, evaluated in the same manner as in Example 1, are substantially equivalent in the degree (degree of isolation-purification of the nucleic acid) to Example 1, and the conditions of this Example show that the washing time is sufficient with 20 seconds and twice repetitions of washing is unnecessary.

Accordingly, it has been shown that the purification of the nucleic acid can be realized easily and rapidly.

The present application claims foreign priority based on Japanese Patent Application Nos. 2003-371783 and 2004-293641, filed Oct. 31, 2003 and Oct. 6, 2004, respectively, the contents of which is incorporated herein by reference.

1. A method for isolating and purifying nucleic acid, comprising the steps of:
(1) passing a sample solution containing a nucleic acid through a nucleic acid adsorbing porous membrane to adsorb the nucleic acid to the nucleic acid adsorbing porous membrane;
(2) passing a washing solution through the nucleic acid adsorbing porous membrane to wash the nucleic acid adsorbing porous membrane while adsorbing the nucleic acid; and
(3) passing an elution solution through the nucleic acid adsorbing porous membrane to desorb the nucleic acid from the nucleic acid adsorbing porous membrane,
wherein the nucleic acid adsorbing porous membrane is a porous membrane capable of adsorbing the nucleic acid by interaction involving substantially no ionic bond, and
a step of drying the nucleic acid adsorbing porous membrane adsorbing the nucleic acid is not included between the washing step (2) and the recovering step (3).

2. The method for isolating and purifying nucleic acid as described in claim 1, wherein the washing solution has surface tension of 3.5 mN/m or less.

3. The method for isolating and purifying nucleic acid as described in claim 2, wherein in the above mentioned each step of (1), (2) and (3), the sample solution containing the nucleic acid, the washing solution or the elution solution is passed through the nucleic acid adsorbing porous membrane under a pressurizing condition.

4. The method for isolating and purifying nucleic acid as described in claim 2, which comprises:
injecting the sample solution containing the nucleic acid, the washing solution or the elution solution in the above mentioned each step of (1), (2) and (3) into one opening of the cartridge for isolation and purification of nucleic acid, in which said cartridge comprises the nucleic acid adsorbing porous membrane in a container having at least two openings;
passing the injected solutions through the interior of the cartridge pressurized by use of a differential pressure generator connected to one opening of the container; and
discharging the injected solutions out of the other opening of the container.

5. The method for isolating and purifying nucleic acid as described in claim 2, wherein a number of times of passing the washing solution through the nucleic acid adsorbing porous membrane adsorbing the nucleic acid to wash the nucleic acid adsorbing porous membrane is once.

6. The method for isolating and purifying nucleic acid as described in claim 2, wherein the steps of passing the washing solution through the porous membrane adsorbing the nucleic acid to wash the nucleic acid adsorbing porous membrane is performed at room temperature.

7. The method for isolating and purifying nucleic acid as described in claim 2, wherein the nucleic acid adsorbing porous membrane is a porous membrane of an organic polymer having a polysaccharide structure.

8. The method for isolating and purifying nucleic acid as described in claim 7, wherein the porous membrane of the organic polymer having the polysaccharide structure is a porous membrane of a mixture of cellulose acetates having different acetyl values.

9. The method for isolating and purifying nucleic acid as described in claim 8, wherein the mixture of cellulose acetates having different acetyl values is a mixture of cellulose triacetate and cellulose diacetate.

10. The method for isolating and purifying nucleic acid as described in claim 9, wherein a mixing ratio (mass ratio) of cellulose triacetate and cellulose diacetate is 99:1 to 1:99.

11. The method for isolating and purifying nucleic acid as described in claim 8, wherein the mixture of cellulose acetates having different acetyl values is a mixture of cellulose triacetate and cellulose monoacetate.

12. The method for isolating and purifying nucleic acid as described in claim 8, wherein the mixture of cellulose acetates having different acetyl values is a mixture of cellulose triacetate, cellulose diacetate and cellulose monoacetate.

13. The method for isolating and purifying nucleic acid as described in claim 8, wherein the mixture of cellulose acetates having different acetyl values is a mixture of cellulose diacetate and cellulose monoacetate.

14. The method for isolating and purifying nucleic acid as described in claim 7, wherein the porous membrane of the organic polymer having the polysaccharide structure is a porous membrane of the organic material containing the saponified cellulose acetate.

15. The method for isolating and purifying nucleic acid as described in claim 14, wherein the saponification degree of the saponified cellulose acetate is 5% or more.

16. The method for isolating and purifying nucleic acid as described in claim 14, wherein the organic material of the saponified cellulose acetate is an organic material of the saponified mixture of cellulose acetates having different acetyl values.

17. The method for isolating and purifying nucleic acid as described in claim 16, wherein the saponification degree of the saponified mixture of cellulose acetate having different acetyle values is 5% or more.

18. The method for isolating and purifying nucleic acid as described in claim 16, wherein the organic material of the saponified mixture of cellulose acetate having different acetyl values is a saponified mixture of cellulose triacetate and cellulose diacetate.

19. The method for isolating and purifying nucleic acid as described in claim 18, wherein the mixing ratio (mass ratio) of cellulose triacetate and cellulose diacetate is 99:1 to 1:99.

20. The method for isolating and purifying nucleic acid as described in claim 16, wherein the organic material of the saponified mixture of cellulose acetate having different acetyl values is a saponified mixture of cellulose triacetate and cellulose monoacetate.

21. The method for isolating and purifying nucleic acid as described in claim 16, wherein the organic material of the saponified mixture of cellulose acetate having different acetyl values is a saponified mixture of cellulose triacetate, cellulose diacetate and cellulose monoacetate.

22. The method for isolating and purifying nucleic acid as described in claim 16, wherein the organic material of the saponified mixture of cellulose acetate having different acetyl values is a saponified mixture of cellulose diacetate and cellulose monoacetate.

23. The method for isolating and purifying nucleic acid as described in claim 14, wherein the porous membrane of the organic material containing the saponified cellulose acetate is a porous membrane where an average hole diameter after saponification reduces compared with that before saponification.

24. The method for isolating and purifying nucleic acid as described in claim 23, wherein a ratio of the average hole diameter after saponification to the average hole diameter before saponification is 0.8 or less.

25. The method for isolating and purifying nucleic acid as described in claim 2, wherein the nucleic acid adsorbing porous membrane is a regenerated cellulose.

26. The method for isolating and purifying nucleic acid as described in claim 2, wherein the nucleic acid adsorbing porous membrane is a porous membrane obtained by treating an organic material having no hydrophilic group to introduce hydrophilic group.

27. The method for isolating and purifying nucleic acid as described in claim 26, wherein the introduction of the hydrophilic group to the organic material having no hydrophilic group comprises combining a graft polymer chain having hydrophilic group with the organic material having no hydrophilic group.

28. The method for isolating and purifying nucleic acid as described in claim 2, wherein the nucleic acid adsorbing porous membrane is a porous membrane obtained by coating the organic material having no hydrophilic group with a material having hydrophilic group to introduce hydrophilic group.

29. The method for isolating and purifying nucleic acid as described in claim 28, wherein the material having hydrophilic group is an organic polymer having hydrophilic group.

30. The method for isolating and purifying nucleic acid as described in claim 2, wherein the nucleic acid adsorbing porous membrane is a porous membrane of inorganic material having hydrophilic group.

31. The method for isolating and purifying nucleic acid as described in claim 2, wherein the nucleic acid adsorbing porous membrane is a porous membrane obtained by treating an inorganic material having no hydrophilic group to introduce hydrophilic group.

32. The method for isolating and purifying nucleic acid as described in claim 31, wherein the introduction of the hydrophilic group to the inorganic material having no hydrophilic group comprises combining a graft polymer chain having the hydrophilic group with inorganic material having no hydrophilic group.

33. The method for isolating and purifying nucleic acid as described in claim 2, wherein the nucleic acid adsorbing porous membrane is a porous membrane obtained by coating the inorganic material having no hydrophilic group with a material having hydrophilic group to introduce hydrophilic group.

34. The method for isolating and purifying nucleic acid as described in claim 33, wherein the material having hydrophilic group is an organic polymer having hydrophilic group.

35. The method for isolating and purifying nucleic acid as described in claim 26, 28, 31 or 33, wherein the hydrophilic group is a hydroxyl group.

36. The method for isolating and purifying nucleic acid as described in claim 2, wherein the nucleic acid adsorbing porous membrane is a porous membrane where the front and back sides are asymmetric.

37. A cartridge for isolating and purifying nucleic acid, comprising a nucleic acid adsorbing porous membrane in a container having at least two openings, the cartridge being used for the method for isolating and purifying a nucleic acid as described in claim 2.

38. A kit for isolating and purifying a nucleic acid, which comprises: a cartridge for isolating and purifying the nucleic acid, comprising a nucleic acid adsorbing porous membrane in a container having at least two openings; and a reagent containing at least a washing solution and an elution solution, the kit being used for the method for isolating and purifying a nucleic acid as described in claim 2.
